(19) United States
(12) Patent Application Publication
(10) Pub. No.: US 2010/0023029 A1
(43) Pub. Date: Jan. 28, 2010

(54) Mesh Device for Immediate Breast Construction and Uses Thereof

(75) Inventor: Leroy Young, Wildwood, MO (US)

Correspondence Address: THOMPSON COBURN LLP, ONE US BANK PLAZA, SUITE 3500, ST LOUIS, MO, 63101, US

(73) Assignee: Bodyaesthetic Research Center, Inc., St. Louis, MO (US)

(21) Appl. No.: 12/495,103

(22) Filed: Jun. 30, 2009

Related U.S. Application Data
(60) Provisional application No. 61/083,036, filed on Jul. 23, 2008.

(51) Int. Cl. A61B 17/08; A61F 2/12

(52) U.S. Cl. 606/151; 623/8

(57) ABSTRACT

An implantable fixation device for maintaining the pre-surgical position of the pectoralis major muscle in relation to the inframammary crease and the anterior axillary line following detachment of the pectoralis major muscle from its medial and inferior origin, such as may be done during immediate breast reconstruction, and methods of use thereof. The device comprises a sheet made of a biocompatible material, such as Vicryl®, that is also elastic and bioabsorbable. The sheet has a number of attachment regions for attaching the sheet to the patient's anatomy. The device may partially cover and constrain a tissue expander or implant, and it may be pre-shaped to further adapt its shape to these surgically implanted devices. The sheet's elasticity allows it to stretch along with the expansion of a tissue expander and yet continue to maintain the position of the pectoralis major muscle. Further, because it is bioabsorbable, the sheet holds the muscle in place just long enough to allow for its self re-attachment without long term concerns.

### CROSS-REFERENCE TO RELATED APPLICATIONS

This application claims priority to U.S. Provisional Patent Application Ser. No. 61/083,036, filed Jul. 23, 2008, and incorporated herein by reference in its entirety.

### STATEMENT REGARDING FEDERALLY SPONSORED RESEARCH OR DEVELOPMENT

Not applicable.

### BACKGROUND OF THE INVENTION

The present invention relates to an implantable fixation device for maintaining the position of the pectoralis major muscle in relation to the inframammary crease and the anterior axillary line following detachment of the pectoralis major muscle from its medial and inferior origin, such as may be done during immediate breast reconstruction, and methods of use thereof.

During immediate breast reconstruction the pectoralis major muscle is detached inferiorly and medially from its origin to place a tissue expander or implant beneath the pectoralis major muscle. Often a tissue expander is first implanted into the patient. Over time, the volume of the tissue expander is increased by adding saline into the tissue expander. Once a patient's body has adapted to the final size and shape of the tissue expander, a permanent implant may be substituted for the tissue expander. In order to insert the tissue expander, the pectoralis major muscle is cut at the sternum, upper costal cartilages, and aponeurosis of the external oblique. If nothing is done after detachment to maintain the muscle's position, the muscle will retract superiorly and laterally towards its distal attachment at the lateral lip of the intertubercular groove of the humerus. Such retraction results in loss of coverage over the tissue expander or implant. Such coverage is necessary to achieve satisfactory expansion and padding of the tissue expander or implant.

Implantable supports have been devised for use in surgical breast procedures. One such support is designed specifically to hold a breast implant in position. (See for example U.S. Ser. No. 11/196,217 filed Aug. 3, 2005 and U.S. Ser. No. 11/564,627 filed Nov. 29, 2006). This support does not address the problem of maintaining the proper position of the pectoralis major muscle and is unsuitable for such use. Further, the support is a permanent prosthesis that permanently remains within the body following its implantation. Safety concerns have been raised because of the permanent nature of the prosthesis in the body. It is unknown whether long term contact between the prosthesis and implant it supports could cause the implant to leak or rupture.

Another type of implantable support used in surgical breast procedures is one made of the biologic material AlloDerm® (LifeCell Corporation, Branchburg, N.J.). AlloDerm® is a tissue matrix prepared from human donor tissue. As such, it is exorbitantly expensive, so much so that at present some insurance carriers will not cover its use. The size of AlloDerm® is limited and thus, multiple pieces must often be attached together. This adds additional difficulty and cost to its use. Although the perimeter of a sheet of AlloDerm® may be cut to form a desired shape, it cannot be configured, pre-shaped, formed, or molded specifically to receive a tissue expander or an implant. Further, AlloDerm® does not possess elastic properties and thus cannot easily stretch or conform to the shape of a tissue expander or an implant, for instance, as when additional volume is added into a tissue expander.

Thus, there is a long felt need in the art to provide for a device that prevents the retraction of the pectoralis major muscle after it has been detached from its origin. Such a fixation device and methods of use thereof would be especially useful following immediate breast reconstruction and have the potential of greatly improving the outcome of patients undergoing such procedure. The device would preferably not be permanent, and thus would not raise concerns about the long term integrity of permanent implants. Among other things, such device can be configurable to receive and constrain a tissue expander or an implant, and also be elastic to additionally conform thereto. Similarly, it is contemplated that the device could also be useful following mastopexy and reduction surgery for shaping and support of internal body structures. Another desirable improvement would be a significant reduction in cost over currently used devices.

### SUMMARY OF THE INVENTION

The present invention is directed to a fixation device for maintaining the position of the pectoralis major muscle. In its preferred embodiment, the fixation device comprises a sheet of biocompatible material configured to maintain a desired position of the pectoralis major muscle following detachment of the muscle from its medial and inferior origin, such as may be done during immediate breast reconstruction. The sheet of biocompatible material is further preferably elastic and bioabsorbable into the body of a patient. The fixation device is also preferably configured to maintain a desired spatial relationship of the pectoralis major muscle with respect to the inframammary crease and the anterior axillary line, such as for example after a tissue expander or an implant is placed in a partial submuscular position beneath the muscle. The fixation device may also be preferably configured to constrain the movement of the tissue expander or implant within the body of a patient, and specifically pre-shaped to receive a tissue expander or implant. The elastic properties of the fixation device may preferably allow for the expansion of a tissue expander.

The present invention is also directed to a fixation device preferably comprising a sheet of biocompatible material that is elastic and is bioabsorbable into the body of a patient; such sheet being configured to comprise a region for attachment to the inferior edge of the pectoralis major muscle, a region for attachment to the lateral edge of the pectoralis major muscle, a region for attachment to the chest wall adjacent to the sternum, a region for attachment to the chest wall along the anterior axillary line, and a region for attachment to the chest wall along the inframammary crease. The fixation device may further preferably comprise a coverage region that covers a tissue expander or an implant, at least in the area inferior to the pectoralis major muscle, when the tissue expander or implant is placed in a partial submuscular position beneath the pectoralis major muscle and the attachment regions of the fixation device are attached to their corresponding body structures.

The present invention is also directed to a method for fixing the position of a pectoralis major muscle that has been detached from its inferior and medial origin and in a preferred embodiment comprises attaching the inferior and lateral edges of the pectoralis major muscle to a sheet of biocompatible material that is elastic and bioabsorbable, and further attaching the sheet to the chest wall so that the pectoralis major muscle remains in a desired position. The sheet of material may be attached to the chest wall adjacent to the sternum, along the anterior axillary line, along the inframammary crease, or attached in any combination thereof.

The present invention is also directed to a method for fixing the position of a pectoralis major muscle that has been detached from its inferior and medial origin by shaping a sheet of biocompatible material so that it is configured to maintain a desired position of the pectoralis major muscle following detachment of the pectoralis major muscle from its medial and inferior origin, wherein the sheet of biocompatible material is preferably elastic and bioabsorbable into the body of a patient; and implanting said sheet into a patient.

### BRIEF DESCRIPTION OF THE SEVERAL VIEWS OF THE DRAWING

FIG. 1 is an anteroposterior view illustrating the fixation device of the invention sutured in place to maintain the proper positioning of the pectoralis major muscle over a tissue expander.

FIG. 2A is a saggital section through approximately the midline of the breast of a patient with a partial submuscular tissue expander or implant illustrating the position of the fixation device over the tissue expander or implant.

FIG. 2B is an anteroposterior view illustrating the position (dashed line 100) of the saggital section of FIG. 2A.

FIG. 3A is a coronal section below the level of the inferior edge of the pectoralis major muscle of a patient with a partial submuscular tissue expander or implant illustrating the position of the device over a tissue expander or implant.

FIG. 3B is an anteroposterior view illustrating the position (dashed line 101) of the coronal section of FIG. 3A.

### DETAILED DESCRIPTION OF THE PREFERRED EMBODIMENT

The present invention is directed to an implantable fixation device for maintaining the position of the pectoralis major muscle in relation to the inframammary crease and the anterior axillary line following detachment of the pectoralis major muscle from its medial and inferior origin, for example such as may be done during immediate breast reconstruction to accommodate the placement of a tissue expander or an implant under the pectoralis major muscle. If nothing is done to maintain the position of the pectoralis major muscle following detachment, the muscle will retract superiorly and laterally. When a tissue expander or implant is present, this will cause the desired coverage of the tissue expander or implant by the muscle to be lost. Further, it is desirable not only to simply maintain the pectoralis muscle in a fixed position, but also to maintain a desired relationship of the muscle to the inframammary crease and the anterior axillary line. The present invention is significant in that it is specifically configured to maintain the muscle in such a position. The present invention is also significant in that it is comprised of a bioabsorbable material that possesses elastic properties and can also be configured to constrain a tissue expander or breast implant.

### A. Structural Material

The present invention provides for a sheet of material that is implanted into the body and provides structural support for fixing internal body structures into place. By sheet, it is meant that the depth or thickness of the structural portion is small relative to its length and width. Because of its use as a structural support, the material should be strong enough, resilient enough, and/or elastic enough to hold its shape and to withstand the forces it will encounter within the body without ripping, tearing, shredding, or the like. It should be easily surgically attachable, such as by suturing, to various internal body parts. In addition, because of its use within the body, the material of the present invention is preferably made of a material that is generally safe for internal implantation such as for example sterile, inert, nonantigenic, nonpyrogenic, etc.

The material is preferably biodegradable or bioabsorbable. Such a material would eliminate the worry of long term contact between the fixation device and an implant. The material may be synthetic or derived from a biological source. Compared to the expense, difficulty and complexity of preparation, quality and consistency issues, and concerns regarding biological contamination of biologically derived materials, synthetic materials may be less expensive, simpler to produce, more consistent, and without concerns regarding biological contamination. Experiments to date have successfully used a VICRYL® mesh. VICRYL® (polyglactin 910) woven or knitted mesh is prepared from a synthetic absorbable copolymer of glycolide and lactide, derived respectively from glycolic and lactic acids. VICRYL® mesh is preferably prepared from uncoated, undyed fiber identical in composition to that used in VICRYL® (polyglactin 910) synthetic absorbable sutures, which have been found to elicit only a mild tissue reaction during absorption. VICRYL® mesh generally persists within the body for about 30 days before it is completely absorbed. During this time, the fixation device serves its purpose of maintaining the position of the pectoralis major muscle. However, over time, the pectoralis major muscle reattaches to the overlying skin flap. Such reattachment takes approximately 14 to 21 days. Once the muscle has formed such attachment, there is no longer a need for a fixation device to maintain the position of the muscle. Therefore, a permanent device to hold the muscle in place is unnecessary and not preferred.

The material is preferably pliable or flexible or the like so that it can be conformed into a desired three dimensional configuration. For example, the sheet of material may wrap around body structures or implanted structures to cover or constrain them. Each patient may have individual dimensions and arrangements of their anatomy that may require adjustments to be made for optimum placement of the fixation device. Thus, it would be undesirable to have a material that is so stiff or non-resilient as to make it difficult for such adjustments. Additionally, the material preferably has elastic qualities such that it is for example stretchable. Such elasticity would further enhance the adjustability of the device to an individual patient.

The elasticity of the material can also be utilized to allow the fixation device to adapt following a surgical procedure. For example, during immediate breast reconstruction a tissue expander may be placed in a partial submuscular position partially below the pectoralis major muscle. Over time following placement of the tissue expander, saline is added to the tissue expander to increase its volume, thus stretching the surrounding tissue. Elasticity of the fixation device allows it to stretch in response to the enlargement of the tissue expander. Thus, a preferred material would be one in which the elasticity can be manipulated to achieve a desired balance between providing enough support and providing enough freedom of movement to accommodate a tissue expander or implant. Experiments to date using different constructions of VICRYL® mesh have been informative. For example, elastic modulus data (see Example 1) indicates that VICRYL® woven mesh has a generally similar elastic modulus in both the machine and cross directions. VICRYL® knitted mesh however is generally more elastic in the machine direction (along the direction of the wale), but less elastic in the cross direction. Therefore, in one embodiment, the device preferably comprises a VICRYL® knitted mesh orientated such that the wale runs vertically or near vertically when a patient is standing. Such an orientation should allow greater expansion of a tissue expander or implant anteriorly and limit expansion laterally or medially because the knitted mesh is less elastic in the cross direction. Further, the greater elasticity in the vertical direction should allow gravity to pull the tissue expander or implant inferiorly, and prevent the tissue expander or implant from being pushed or squeezed superiorly, thus giving the breast a natural shape.

### B. Configuration of the Fixation Device

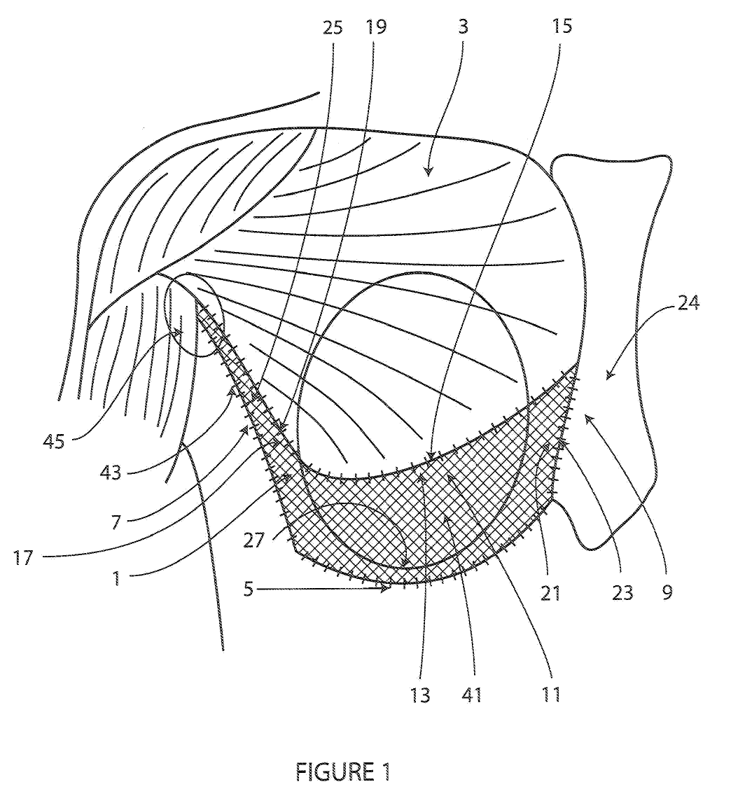

FIGS. 1 through 3 show an exemplary embodiment of the present inventive fixation device. In general, the fixation device of the present invention comprises a sheet of a structural material that is attached to the pectoralis major muscle as well as to other surrounding internal body structures such as, for example, the chest wall.

FIG. 1 is an anteroposterior view of a preferred embodiment of the present invention. As can be seen, the embodiment comprises a sheet of structural material 1 configured to hold the pectoralis majoris muscle 3 in place in relationship to the level of the inframammary crease 5 and the anterior axillary line 7 following detachment of the pectoralis major muscle from its medial and inferior origin 9. Further, the fixation device is preferably configured so that it may cover a tissue expander or implant 11 inserted submuscularly under the pectoralis major muscle, such as may be implanted during immediate breast reconstruction.

The sheet of structural material 1 is configured to comprise multiple regions for attachment to various internal structures of the body. These attachment regions are apparent in the anteroposterior view of FIG. 1. A first attachment region 13 is configured to attach to the inferior edge of the pectoralis major muscle 15. A second attachment region 17 is configured to attach to the lateral edge of the pectoralis major muscle 19. A third attachment region 21 is configured to attach to the chest wall adjacent to the sternum 23 (sternum itself 24). A fourth attachment region 25 is configured to attach to the chest wall along the anterior axillary line 7. A fifth attachment region 27 is configured to attach to the chest wall along the inframammary crease 5. Thus, when affixed to the body in a manner corresponding to the intended area of attachment for each attachment region, the fixation device will be oriented in a patient's body as depicted in FIG. 1. Together, these attachments are expected to reliably prevent the pectoralis major muscle from retracting superiorly and laterally toward the distal attachment of the muscle at the humerus.

The attachment regions may be indistinguishable from other portions of the sheet of material except for their position on the sheet. That is, in one embodiment, their construction does not vary from the other portions of the sheet. In other embodiments however, one or more of the attachment regions may be distinguishable. In one embodiment, the material at the perimeter of the sheet coextensive with the attachment regions, can be folded over. In another embodiment, the material of the attachment regions can be made thicker to distinguish it from other portions of the sheet. In a further embodiment, the attachment regions can comprise a dedicated material other than the material used to construct the sheet of material. One of skill in the art will realize that numerous other such variations in construction are possible. Such variations of the attachment regions may be advantageous such as for example by providing additional support to prevent detachment of the device, by indicating the orientation of the device to the surgeon, or by providing a more optimal attachment point during surgery to secure the device to the body.

The length of the first attachment region is preferably between about 14 centimeters and about 18 centimeters, more preferably about 16 centimeters. The length of the second attachment region is preferably between about 4 centimeters and about 8 centimeters, and more preferably about 6 centimeters. The length of the third attachment region is preferably between about 11 centimeters and about 15 centimeters, and more preferably about 13 centimeters. The length of the fourth attachment region is preferably between about 10 centimeters and about 14 centimeters, and more preferably about 12 centimeters. The length of the fifth attachment region is preferably between about 7 centimeters and about 11 centimeters, and more preferably about 9 centimeters. One of skill in the art will recognize that these lengths will depend on the anatomy of the patient undergoing operation and are thus subject to variation. It is contemplated that fixation devices may be provided prior to surgery in one or several standard sizes, but that the exact dimension of the device may be customized by cutting or other such sizing of the sheet prior to use. Further, one of skill in the art will recognize that the boundaries between these regions are not definite and therefore the regions may be considered to overlap.

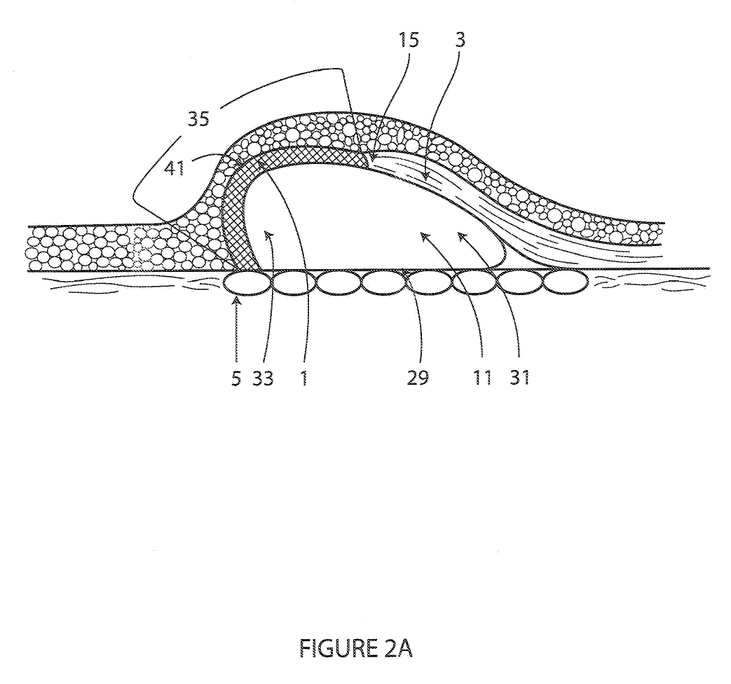

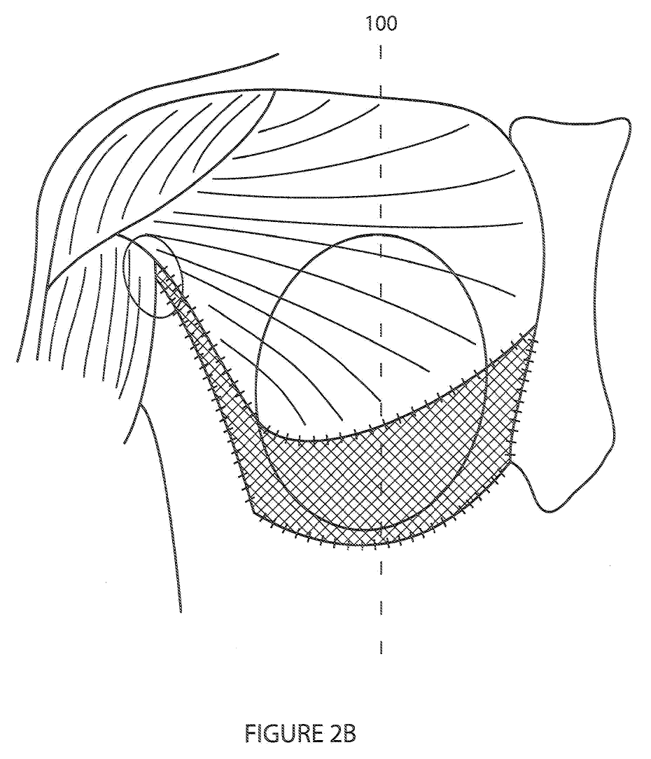

FIG. 2A is a saggital section through approximately the midline of the breast of a patient with a partial submuscular tissue expander or implant illustrating the position of the fixation device over the tissue expander or implant. FIG. 2B is an anteroposterior view illustrating the position (dashed line 100) of the saggital section of FIG. 2A. As can be seen, the tissue expander or implant 11 is positioned against the chest wall 29 and is partially covered by the pectoralis major muscle 3. The sheet of structural material 1 of the fixation device is attached on one side to the inferior edge of the pectoralis major muscle 15 and on the other side to the chest wall at the level of the inframammary crease 5. The pectoralis major muscle covers the superior portion 31 of the tissue expander or implant, but does not cover the inferior portion 33. Thus, the fixation device bridges this gap 35 between the inferior edge of the pectoralis major muscle and the inframammary crease and covers the inferior portion of the tissue expander or implant. The fixation device may come into direct communication with the tissue expander or implant. However, as previously discussed, the long term implications of this contact on the integrity of a permanent implant are not deemed worthy of concern because of the transient, bioabsorbable nature of the fixation device.

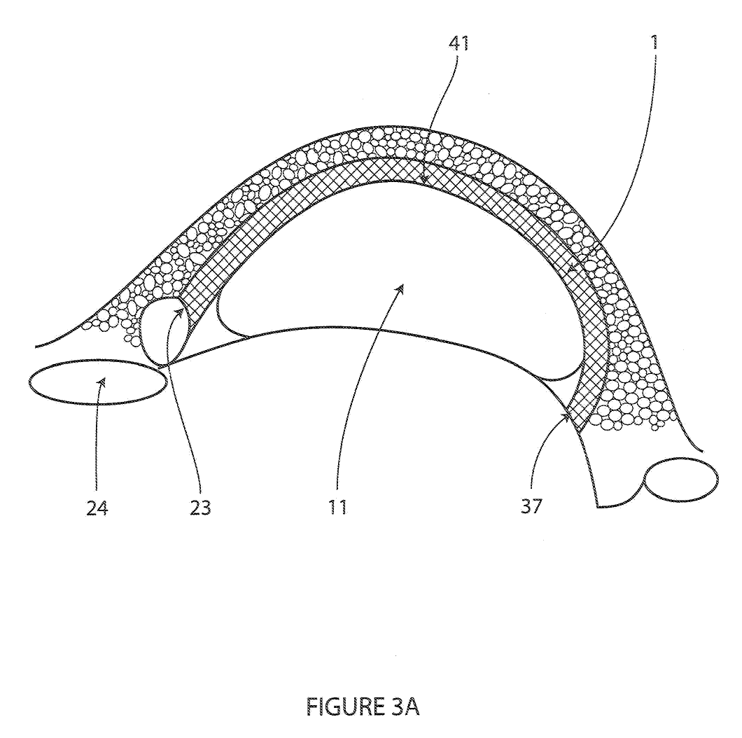

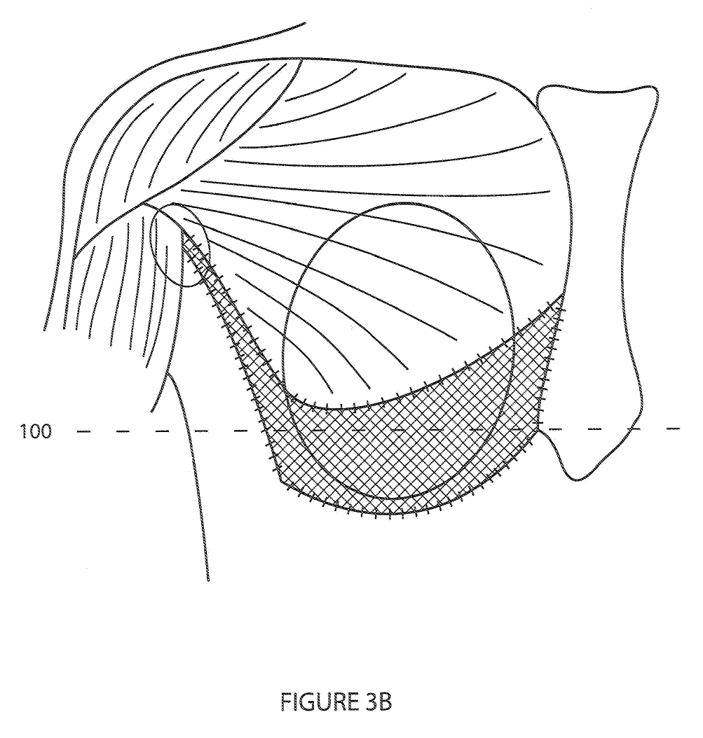

FIG. 3A is a coronal section through the chest of a patient with a partial submuscular tissue expander or implant at a level inferior to the pectoralis major muscle. FIG. 3B is an anteroposterior view illustrating the position (dashed line 101) of the coronal section of FIG. 3A. At this level, the pectoralis major muscle (not shown) no longer provides coverage for the tissue expander or implant 11. This figure further illustrates how the sheet of structural material 1 of the fixation device covers the region of the tissue expander or implant inferior to the inferior edge of the pectoralis major muscle. As can been seen, the device provides coverage of the tissue expander or implant from a point of attachment 23 adjacent to the sternum 24 where the pectoralis major muscle was detached from its origin, laterally across the tissue expander or implant to a point of attachment 37 on the opposite side of the tissue expander or implant along the anterior axillary line.

In the preferred embodiment illustrated in FIGS. 1 through 3, the fixation device comprises a coverage region 41 that is configured to cover at least a portion of a tissue expander or implant that is inferior to the pectoralis major muscle. As can be seen in FIGS. 2 and 3, the shape of the sheet of the fixation device 1 bends or curves to conform to the shape of the underlying tissue expander or implant 11. As previously noted, the ability of the fixation device to conform to the shape of the tissue expander or implant is at least in part due to the pliable or flexible nature of the material comprising the device. In addition, the elastic or stretchable nature of the material further enhances its ability to conform to the shape of the tissue expander or implant as well as to other body structures.

The coverage area 41 may also be pre-shaped, such as including a curvature, to receive a tissue expander or implant. Pre-shaping of the coverage area may further enhance the fit between the fixation device and tissue expander or implant and increase the ability of the device to constrain the tissue expander or implant in addition to fixing the pectoralis major muscle in place. The pre-shaping of the coverage area may be very specific so that a fixation device of a certain shape would be ideally suited for a specific tissue expander or implant of a given shape or volume while also allowing further individual customization to accommodate its use with different anatomies. In addition, the fixation device may be pre-shaped in any way in order to enhance its configuration so the it conforms to its implantation and attachment within the patient's body.

As can be seen in FIG. 1, the region of coverage 41 is a region that is partially defined by several of the aforedescribed regions of attachment. The region of coverage is defined along its superior edge when implanted in a patient by the attachment region configured to attach to the inferior edge of the pectoralis major muscle 13. The region of coverage is defined along its medial edge when implanted in a patient by the attachment region configured to attach to the chest wall adjacent to the sternum 21. The region of coverage is defined along its inferior edge when implanted in a patient by the attachment region configured to attach to the chest wall along the inframammary crease 27.

FIG. 1 also illustrates an additional extending region 43 of the sheet that is configured to extend laterally and superiorly toward the axillary fat pad 45. This region is partially defined on its medial edge when implanted in a patient by the attachment region configured to attach to the lateral edge of the pectoralis major muscle 19 and on its lateral edge when implanted into a patient by the attachment region configured to attach to the chest wall along the anterior axillary line 25. This extending region provides additional containment for a tissue expander or implant, preventing lateral displacement. The extending region may be contiguous and/or overlap in area with the region covering 41 the tissue expander or implant 11. Thus, the extending region may be pre-shaped in the same manner as, and/or in conjunction with, the region covering the tissue expander or implant to provide the desired conformation of the fixation device to the tissue expander or implant.

### C. Method of Preventing Retraction of the Pectoralis Major Muscle

The method of the current invention is directed to securing the pectoralis major muscle following detachment from its inferior and lateral origin. Such detachment may be required, for example, during immediate breast reconstruction so that a tissue expander or implant may be inserted under the muscle. In particular, the method of the current invention is directed to securing the pectoralis major muscle to the fixation device of the invention. Attachment of the fixation device to the muscle and to the chest wall may occur by any means recognized in the medical arts. In a preferred use, the fixation device is attached by suturing. The fixation device not only secures the muscle, but also maintains the desired relationship of the pectoralis major muscle to the inframammary crease and the anterior axillary line. This is achieved because the fixation device is specifically configured, as previously described, to attach to lateral and inferior regions of the muscle and to other structures within the body capable of maintaining the desired position of the muscle.

In an embodiment of the method where a tissue expander or implant is implanted in a patient, the fixation device covers at least a part of the tissue expander or implant. Thus, the device may come into direct communication with the tissue expander or implant and therefore may constrain the movement of the tissue expander or implant. In a preferred embodiment, the fixation device is pre-shaped to receive the tissue expander or implant. Although prior supports have been used to constrain an implant, the current invention constrains the tissue expander or implant while also serving to fix the pectoralis major muscle in place.

Another embodiment of the method of the current invention involves shaping, cutting, assembling, constructing, or the like, a sheet of material of the type used for the fixation device such that it is configured to comprise the various regions of attachment, coverage region, and extending region as previously described.

### EXAMPLES

### Example 1

Elastic modulus data for VICRYL® knitted and woven mesh.

M4R tensile GL1″ XH5″/min

| | |
|---|---|
| Sample Identification: | VICRYLMD |
| Test Method Number: | 61 |
| Test Date: | Wednesday, March 26, 2008 |
| Interface Type: | 4200 |
| Crosshead Speed: | 5.0000 in/min |
| Sample Rate (pts/secs): | 20.0000 |
| Temperature: | 73 F. |
| Humidity (%): | 50 |
| Grip Distance: | 1.0000 in |
| Specimen G.L.: | 1.0000 in |

TABLE 1a
Results Vicryl Knitted Mesh Machine (wale) direction (Thickness = 0.0077″)

| | | | | | | Modular | Modulus | Modulus | % Strain |
|---|---|---|---|---|---|---|---|---|---|
| | Max. | Load/Width at | Max. | Max. | Max. | Secant | 0.5-1.5% | 70-100% | @ 1#load |
| | Load | Max. Load | Disp. | Strs | Strn | (.2-1#) | strain | (usr lims) | EASL1 |
| | (lbf) | (lbf/in) | (in) | (psi) | (%) | (psi) | (psi) | (psi) | (%) |
| 1 | 16.1 | 16.1 | 1.173 | 2092.2 | 117.3 | 343 | 1026 | 2811 | 32.7 |
| 2 | 18.3 | 18.3 | 1.218 | 2372.7 | 121.8 | 364 | 989 | 2916 | 31.2 |
| 3 | 18.7 | 18.7 | 1.691 | 2426.0 | 169.1 | 204 | 540 | 1751 | 56.9 |
| 4 | 19.0 | 19.0 | 1.340 | 2474.0 | 134.0 | 305 | 563 | 2654 | 37.8 |
| 5 | 13.3 | 13.3 | 1.382 | 1724.7 | 138.2 | 229 | 515 | 1912 | 52.0 |
| 6 | 16.5 | 16.5 | 1.501 | 2146.8 | 150.1 | 219 | 454 | 1910 | 54.4 |
| Mean | 17.0 | 17.0 | 1.384 | 2206.1 | 138.4 | 277 | 681 | 2326 | 44.2 |
| S.D. | 2.2 | 2.2 | 0.191 | 281.5 | 19.1 | 69 | 256 | 522.5 | 11.6 |
| C.V. | 12.8 | 12.8 | 13.789 | 12.8 | 13.8 | 25 | 38 | 22.47 | 26.2 |
| Minimum | 13.3 | 13.3 | 1.173 | 1724.7 | 117.3 | 204 | 454 | 1751 | 31.2 |
| Maximum | 19.0 | 19.0 | 1.691 | 2474.0 | 169.1 | 364 | 1025 | 2916 | 56.9 |

TABLE 1b
Results Vicryl Knitted Mesh Machine (wale) direction (Thickness = 0.0077″)

| | % Strain | % Strain | % Strain @ | Stiffness2 | Stiffness3 | Stiffness70 | StiffnessF |
|---|---|---|---|---|---|---|---|
| | @ 2# | @ 5# | 10# | Load/Width | Load/Width | Load/Width | Load/Width |
| | EASL2 | EASL5# | EASL10# | LASE@20% | LASE@33% | LASE@70% | LASE@100% |
| | (%) | (%) | (%) | (lbf/in) | (lbf/in) | (lbf/in) | (lbf/in) |
| 1 | 47.7 | 67.265 | 91.569 | 0.627 | 1.029 | 5.50 | 11.994 |
| 2 | 46.7 | 65.424 | 88.541 | 0.642 | 1.094 | 5.95 | 12.689 |
| 3 | 73.2 | 95.215 | 125.010 | 0.335 | 0.489 | 1.73 | 5.776 |
| 4 | 52.5 | 71.134 | 95.660 | 0.529 | 0.836 | 4.78 | 10.915 |
| 5 | 69.2 | 91.583 | 118.587 | 0.372 | 0.557 | 2.07 | 6.483 |
| 6 | 70.4 | 91.680 | 119.364 | 0.344 | 0.517 | 1.97 | 6.381 |
| Mean | 59.9 | 80.383 | 106.455 | 0.475 | 0.754 | 3.67 | 9.040 |
| S.D. | 12.2 | 13.816 | 16.230 | 0.143 | 0.269 | 1.93 | 3.157 |
| C.V. | 20.4 | 17.188 | 15.246 | 30.0 | 35.720 | 53.2 | 34.919 |
| Minimum | 46.7 | 65.424 | 88.541 | 0.335 | 0.489 | 1.73 | 5.776 |
| Maximum | 73.2 | 95.215 | 128.010 | 0.642 | 1.094 | 5.95 | 12.689 |

M4B tensile GL1″ XH5″/min

| | |
|---|---|
| Sample Identification: | VICRYLXK |
| Test Date: | Wednesday, March 26, 2008 |
| Test Method Number: | 61 |
| Interface Type: | 4200 |
| Crosshead Speed: | 5.0000 in/min |
| Sample Rate (pts/secs): | 20.0000 |
| Temperature: | 73 F. |
| Humidity (%): | 50 |
| Grip Distance: | 1.0000 in |
| Specimen G.L.: | 1.0000 in |

TABLE 2a
Results Vicryl Knitted Mesh X-direction (0.0045 in thickness)

| | | | | | | | | Modulus | % |
|---|---|---|---|---|---|---|---|---|---|
| | | Load/Width | | | | Modular | Modulus | 70-100% | Strain @ |
| | Max. | at Max. | Max. | Max. | Max. | Secant | 0.5-1.5% | (usr | 1#load |
| | Load | Load | Disp. | Strs | Strn | (.2-1#) | strain | lims) | EASL1 |
| | (lbf) | (lbf/in) | (in) | (psi) | (%) | (psi) | (psi) | (psi) | (%) |
| 1 | 50.1 | 50.1 | 0.709 | 6682.7 | 70.9 | 9183 | 4669 | — | 2.40 |
| 2 | 50.5 | 50.5 | 0.629 | 6740.0 | 62.9 | 8353 | 1199 | — | 3.01 |
| 3 | 50.0 | 50.0 | 0.693 | 6668.0 | 69.3 | 7960 | 2306 | — | 2.84 |
| 4 | 52.2 | 52.2 | 0.744 | 6958.7 | 74.4 | 7871 | 1570 | — | 2.93 |
| 5 | 47.5 | 47.5 | 0.640 | 6328.0 | 64.0 | 6387 | 3214 | — | 2.92 |
| 6 | 54.0 | 54.0 | 0.696 | 7194.7 | 69.6 | 9332 | 2555 | — | 2.61 |
| Mean | 50.7 | 50.7 | 0.685 | 6762.0 | 68.5 | 8181 | 2586 | | 2.79 |
| S.D. | 2.2 | 2.2 | 0.043 | 293.2 | 4.3 | 1070 | 1246 | | 0.232 |
| C.V. | 4.3 | 4.3 | 6.313 | 4.3 | 6.3 | 13 | 48 | | 8.34 |
| Minimum | 47.5 | 47.5 | 0.629 | 6328.0 | 62.9 | 6387 | 1199 | | 2.40 |
| Maximum | 54.0 | 54.0 | 0.744 | 7194.7 | 74.4 | 9332 | 4669 | | 3.01 |

TABLE 2b
Results Vicryl Knitted Mesh X-direction 0.0045 in thickness

| | % | % | | | | | | |
|---|---|---|---|---|---|---|---|---|
| | Strain | Strain | % Strain | Stiffness1 | Stiffness2 | Stiffness3 | Stiffness70 | StiffnessF |
| | @ 2# | @ 5# | @ 10# | Load/Width | Load/Width | Load/Width | Load/Width | Load/Width |
| | EASL2 | EASL5# | EASL10# | LASE@10% | LASE@20% | LASE@33% | LASE@70% | LASE@100% |
| | (%) | (%) | (%) | (lbf/in) | (lbf/in) | (lbf/in) | (lbf/in) | (lbf/in) |
| 1 | 5.1 | 9.776 | 14.590 | 10.0 | 14.6 | 24.415 | 49.7 | — |
| 2 | 4.6 | 8.305 | 12.395 | 10.0 | 17.6 | 27.939 | 7.39 | — |
| 3 | 5.4 | 9.816 | 14.529 | 10.0 | 14.6 | 24.282 | 21.4 | — |
| 4 | 5.3 | 9.381 | 14.079 | 10.0 | 14.9 | 24.189 | 49.5 | — |
| 5 | 5.3 | 9.786 | 14.538 | 10.0 | 14.8 | 24.695 | — | — |
| 6 | 4.7 | 8.716 | 13.040 | 10.0 | 16.4 | 26.454 | 47.2 | — |
| Mean | 5.1 | 9.297 | 13.862 | 10.0 | 15.3 | 25.329 | 0 | |
| S.D. | 0.3 | 0.643 | 0.928 | 0 | 1.22 | 1.530 | 0 | |
| C.V. | 6.7 | 6.916 | 6.694 | 0 | 7.86 | 6.042 | 0 | |
| Minimum | 4.6 | 8.305 | 12.395 | 10.0 | 14.6 | 24.189 | 0 | |
| Maximum | 5.4 | 9.816 | 14.590 | 10.0 | 17.6 | 27.939 | 0 | |

M4B tensile GL1″ XH5″/min

| | |
|---|---|
| Sample Identification: | VICRYLXD |
| Test Date: | Tuesday, March 25, 2008 |
| Test Method Number: | 61 |
| Interface Type: | 4200 |
| Crosshead Speed: | 5.0000 in/min |
| Sample Rate (pts/secs): | 20.0000 |
| Temperature: | 73 F. |
| Humidity (%): | 50 |
| Grip Distance: | 1.0000 in |
| Specimen G.L.: | 1.0000 in |

TABLE 3a
Results Vicryl woven mesh folding direction of 0.0047 in thickness

| | | | | | | | | Modulus | |
|---|---|---|---|---|---|---|---|---|---|
| | | Load/Width | | | | Modular | Modulus | 70-100% | % Strain @ |
| | | at Max. | Max. | Max. | Max. | Secant | 0.5-1.5% | (usr | 1#load |
| | Max. Load | Load | Disp. | Strs | Strn | (.2-1#) | strain | lims) | EASL1 |
| | (lbf) | (lbf/in) | (in) | (psi) | (%) | (psi) | (psi) | (psi) | (%) |
| 1 | 52.9 | 52.9 | 0.376 | 11128.4 | 37.6 | 52093 | 128332 | 710.8 | 0.595 |
| 2 | 57.0 | 57.0 | 0.408 | 11993.7 | 40.8 | 47624 | 118970 | — | 0.463 |
| 3 | 54.8 | 54.8 | 0.429 | 11528.4 | 42.9 | 33411 | 67445 | — | 0.821 |
| 4 | 55.6 | 55.6 | 0.412 | 11709.5 | 41.2 | 34618 | 84426 | — | 0.578 |
| 5 | 56.5 | 56.5 | 0.379 | 11890.5 | 37.9 | 48187 | 112372 | — | 0.437 |
| Mean | 55.3 | 55.3 | 0.401 | 11650.1 | 40.1 | 43187 | 102309 | 0 | 0.579 |
| S.D. | 1.6 | 1.6 | 0.023 | 341.4 | 2.3 | 8558 | 25452 | 0 | 0.152 |
| C.V. | 2.9 | 2.9 | 5.712 | 2.9 | 5.7 | 20 | 25 | 0 | 26.3 |
| Minimum | 52.9 | 52.9 | 0.376 | 11128.4 | 37.6 | 33411 | 67445 | 0 | 0.437 |
| Maximum | 57.0 | 57.0 | 0.429 | 11993.7 | 42.9 | 52093 | 128332 | 0 | 0.821 |

TABLE 3b
Results Vicryl woven mesh folding direction of 0.0047 in thickness

| | % Strain | % Strain | % Strain @ | Stiffness2 | Stiffness3 | Stiffness70 | StiffnessF |
|---|---|---|---|---|---|---|---|
| | @ 2# | @ 5# | 10# | Load/Width | Load/Width | Load/Width | Load/Width |
| | EASL2 | EASL5# | EASL10# | LASE@20% | LASE@33% | LASE@70% | LASE@100% |
| | (%) | (%) | (%) | (lbf/in) | (lbf/in) | (lbf/in) | (lbf/in) |
| 1 | 0.8 | 1.241 | 2.168 | 37.9 | 51.497 | 6.43 | 7.446 |
| 2 | 0.6 | 1.151 | 2.181 | 37.1 | 51.666 | 17.4 | — |
| 3 | 1.2 | 1.748 | 3.091 | 35.5 | 48.254 | — | — |
| 4 | 0.9 | 1.551 | 2.996 | 35.9 | 49.099 | — | — |
| 5 | 0.6 | 1.205 | 2.200 | 37.8 | 52.560 | — | — |
| Mean | 0.8 | 1.379 | 2.527 | 36.9 | 50.616 | 0 | 0.000 |
| S.D. | 0.2 | 0.258 | 0.473 | 1.10 | 1.840 | 0 | 0.000 |
| C.V. | 27.8 | 18.739 | 18.715 | 2.98 | 3.635 | 0 | 0.000 |
| Minimum | 0.6 | 1.151 | 2.168 | 35.5 | 48.254 | 0 | 0.000 |
| Maximum | 1.2 | 1.748 | 3.091 | 37.9 | 52.560 | 0 | 0.000 |

What is claimed:

1. A fixation device for temporarily maintaining the position of a patient's pectoralis major muscle sufficiently to induce its self reattachment after its partial surgical detachment, the device comprising a sheet of biocompatible and bioabsorbable material configured for surgical implantation and attachment between the pectoralis major muscle and its medial and inferior origins, wherein the device is sufficiently sized to maintain a desired spatial relationship of the pectoralis major muscle with respect to the inframammary crease and the anterior axillary line.

2. The fixation device of claim 1 wherein the sheet is sufficiently elastic to allow for its use with a tissue expander or implant and wherein the device is configured to maintain a desired spatial relationship of the pectoralis major muscle with respect to the inframammary crease and the anterior axillary line after the tissue expander or implant is placed in a partial submuscular position beneath the pectoralis major muscle.

3. The fixation device of claim 2 wherein the device is configured to constrain the movement of the tissue expander or implant within the patient's body and wherein the elasticity of the sheet of biocompatible material allows for expansion of the tissue expander.

4. The fixation device of claim 2 wherein the sheet of biocompatible material is pre-shaped to receive the tissue expander or the implant so that it constrains the movement of the tissue expander or the implant.

5. The fixation device of claim 1 wherein the sheet comprises a mesh.

6. The fixation device of claim 5 wherein the mesh is at least partially made of a knitted or woven Vicryl® mesh.

7. A fixation device for temporarily maintaining the position of a patient's pectoralis major muscle sufficiently to induce its self reattachment after its partial surgical detachment, the device comprising a sheet of material that is biocompatible, elastic, bioabsorbable and configured to comprise:
a first attachment region for attachment to the inferior edge of the pectoralis major muscle;
a second attachment region for attachment to the lateral edge of the pectoralis major muscle;
a third attachment region for attachment to the chest wall adjacent to the sternum;
a fourth attachment region for attachment to the chest wall along the anterior axillary line; and
a fifth attachment region for attachment to the chest wall along the inframammary crease.

8. The fixation device of claim 7 wherein one or more of said attach regions is defined from other regions of the device by one or more of the following: folding over of the sheet of material; differing in thickness or construction; or comprising a material different from the material used to form other regions of the device.

9. The fixation device of claim 7 wherein the sheet further comprises a coverage region adapted to cover at least in the area inferior to the pectoralis major muscle an underlying, surgically implanted tissue expander or implant when the tissue expander or implant is placed in a partial submuscular position beneath the pectoralis major muscle.

10. The fixation device of claim 9 wherein said coverage region is defined on three sides by said first attachment region, said third attachment region, and said fifth attachment region.

11. The fixation device of claim 9 wherein said coverage region is pre-shaped to receive said tissue expander or implant so that is constrains the movement of the tissue expander or the implant.

12. The fixation device of claim 7 wherein the sheet of material further comprises an extending region that, when the fixation device is implanted into a patient, is configured to extend laterally and superiorly toward an axillary fat pad, wherein said extending region constrains the lateral movement of the tissue expander or implant.

13. The fixation device of claim 7 wherein the elasticity of the sheet is sufficient to allow for expansion of the tissue expander following surgical placement of the tissue expander and the fixation device into a patient.

14. The fixation device of claim 7 wherein the sheet is a mesh.

15. The fixation device of claim 14 wherein the mesh is at least partially made of a knitted or woven Vicryl® mesh.

16. A method for temporarily maintaining the position of a patient's pectoralis major muscle sufficiently to induce its self reattachment after its partial surgical detachment along its inferior and lateral edges, the method comprising:
a) attaching the inferior and lateral edges of the pectoralis major muscle to a sheet of material that is biocompatible, elastic, and bioabsorbable; and
b) attaching said sheet to the chest wall so that the pectoralis major muscle remains substantially in its pre-surgical position.

17. The method of claim 16 wherein the sheet is attached to the chest wall adjacent to the sternum, along an anterior axillary line, along an inframammary crease, or in any combination thereof.

18. The method of claim 16 further comprising implanting a tissue expander or an implant in a partial submuscular position partially beneath the pectoralis major, such as during immediate breast reconstruction.

19. The method of claim 16 wherein the sheet comprises a knitted Vicryl® mesh;
wherein the sheet is orientated and attached during surgery of a patient such that the direction of the wale of the knitted Vicryl® mesh is vertical or near vertical in the patient when the patient is standing upright.

20. A method for temporarily maintaining the position of a patient's pectoralis major muscle sufficiently to induce its self reattachment after its partial surgical detachment along its inferior and lateral edges, the method comprising:
(a) shaping a sheet of material so that it is configured to maintain a pre-surgical position of the pectoralis major muscle following detachment of the pectoralis major muscle from its medial and inferior origin, wherein the sheet of material is biocompatible, elastic, and bioabsorbable; and
(b) implanting the sheet into a patient's body.

21. The method of claim 20 wherein the sheet comprises: a first attachment region for attachment to the inferior edge of the pectoralis major muscle; a second attachment region for attachment to the lateral edge of the pectoralis major muscle; a third attachment region for attachment to the chest wall adjacent to the sternum; a fourth attachment region for attachment to the chest wall along the anterior axillary line; and a fifth attachment region for attachment to the chest wall along the inframammary crease;
wherein the step of implanting further comprises attaching the sheet to the chest wall adjacent to the sternum, along an anterior axillary line, along an inframammary crease, or in any combination thereof.

22. The method of claim 20 further comprising implanting a tissue expander or an implant in a partial submuscular position partially beneath the pectoralis major, such as during immediate breast reconstruction.

23. The method of claim 20 wherein the method further comprises pre-shaping the sheet of biocompatible material to receive the tissue expander or the implant so that the sheet may constrain the movement of the tissue expander or the implant.

24. The method of claim 20 wherein the sheet comprises a knitted Vicryl® mesh;
wherein the sheet is orientated and attached during surgery of a patient such that the direction of the wale of the knitted Vicryl® mesh is vertical or near vertical in the patient when the patient is standing upright.
